(19) United States
(12) Patent Application Publication
(10) Pub. No.: US 2016/0228280 A1
(43) Pub. Date: Aug. 11, 2016

(54) SYSTEM AND METHOD FOR INVASIVE AND NON-INVASIVE HEAD FIXATION

(71) Applicant: pro med instruments GmbH, Freiburg im Breisgau (DE)

(72) Inventors: Matthias E. Schuele, Freiburg (DE); Bernd Harter, Freiburg (DE)

(21) Appl. No.: 15/016,676

(22) Filed: Feb. 5, 2016

Related U.S. Application Data
(60) Provisional application No. 62/112,427, filed on Feb. 5, 2015.

(51) Int. Cl. A61F 5/37; A61G 13/12

(52) U.S. Cl. CPC A61F 5/3707; A61G 13/121

(57) ABSTRACT

A head fixation device includes a curved frame connectable to a base unit, which is connectable to a patient support device such as an operating table. The frame is connectable to the base unit by way of a swivel adapter, and an interface member that connects with the swivel adapter includes a recess configured to engage the frame. A plurality of stabilization assemblies is connectable to the frame, either directly or by way of one or more support arms. Each of the stabilization assemblies is configurable with an invasive stabilizing structure such as a pin, or a non-invasive stabilizing structure such as a pad.

### PRIORITY

This application claims priority to U.S. Provisional Patent Application Ser. No. 62/112,427, filed Feb. 5, 2015, entitled “System and Method for Invasive and Non-Invasive Head Fixation,” the disclosure of which is incorporated by reference herein.

### BACKGROUND

The systems and methods disclosed pertain to the field of patient stabilization, and in particular head and neck stabilization using head fixation devices (hereinafter referred to as “HFDs” or “HFD” in singular).

HFDs are sometimes used during a variety of surgical and other medical procedures, for example during head or neck surgery or testing where it would be desirable to securely support a patient's head in a certain position. The HFDs described herein are, in some versions, particularly suitable for use with patients having weak cranial bone structure and/or smaller head size, e.g., pediatric patients. However, in some versions the HFDs described herein may also be used or adapted for use with patients having normal or fully developed cranial bone structure and/or head size.

While a variety of HFDs have been made and used, it is believed that no one prior to the inventor(s) has made or used the devices, systems, and methods as described herein. Other aspects, features, and techniques within the scope of the present disclosure will become more apparent to those of ordinary skill in the art from the following description taken in conjunction with the drawings.

### BRIEF DESCRIPTION OF THE DRAWINGS

FIG. 1 depicts a perspective view of an exemplary patient stabilization system.

FIG. 2 depicts a perspective view of a HFD, swivel adapter, and interface adapter of the patient stabilization system of FIG. 1, with a configuration where a frame of the HFD is adjusted about the connection with the interface adapter.

FIG. 3 depicts a perspective view of another exemplary configuration for the HFD, swivel adapter, and interface adapter of FIG. 1, using a non-invasive setup with a plurality of gel pads and multiple support arms added.

FIG. 4 depicts a perspective view of another exemplary configuration for the

HFD, swivel adapter, and interface adapter of FIG. 3, using an invasive and non-invasive setup with a plurality of gel pads and multiple pins.

FIG. 5 depicts a perspective view of another exemplary configuration for the

HFD, swivel adapter, and interface adapter of FIG. 4, with the support arms having pins.

FIG. 6 depicts a perspective view of the frame and adapter of the HFD, swivel adapter, and interface adapter of the patient stabilization system of FIG. 1.

FIG. 7 depicts a front view of the swivel adapter and interface adapter of the patient stabilization system of FIG. 1.

FIG. 8 depicts a side cross-section view of the swivel adapter and interface adapter of the patient stabilization system of FIG. 1.

FIG. 9 depicts a perspective view of one of the support arms of the patient stabilization system of FIG. 5.

FIG. 10 depicts a partial perspective view of an actuator assembly of the support arm of FIG. 9.

FIG. 11 depicts a side view of a stabilization assembly connected with a pin.

FIG. 12 depicts a side cross-section view of the stabilization assembly and pin of FIG. 11.

The drawings are not intended to be limiting in any way, and it is contemplated that various embodiments of the present disclosure may be carried out in a variety of other ways, including those not necessarily depicted in the drawings. The accompanying drawings incorporated in and forming a part of the specification illustrate several aspects, and together with the description serve to explain the principles of the present disclosure; it being understood, however, that the scope of the present disclosure is not limited to the precise arrangements shown.

### DETAILED DESCRIPTION

The following description of certain embodiments should not be used to limit the scope of the present disclosure. Other examples, features, aspects, embodiments, and advantages will become apparent to those skilled in the art from the following description. As will be realized, various aspects of the present disclosure may take alternate forms, or have alternate or additional embodiments, without departing from the scope of the present disclosure. Accordingly, the drawings and descriptions should be regarded as illustrative in nature and not restrictive.

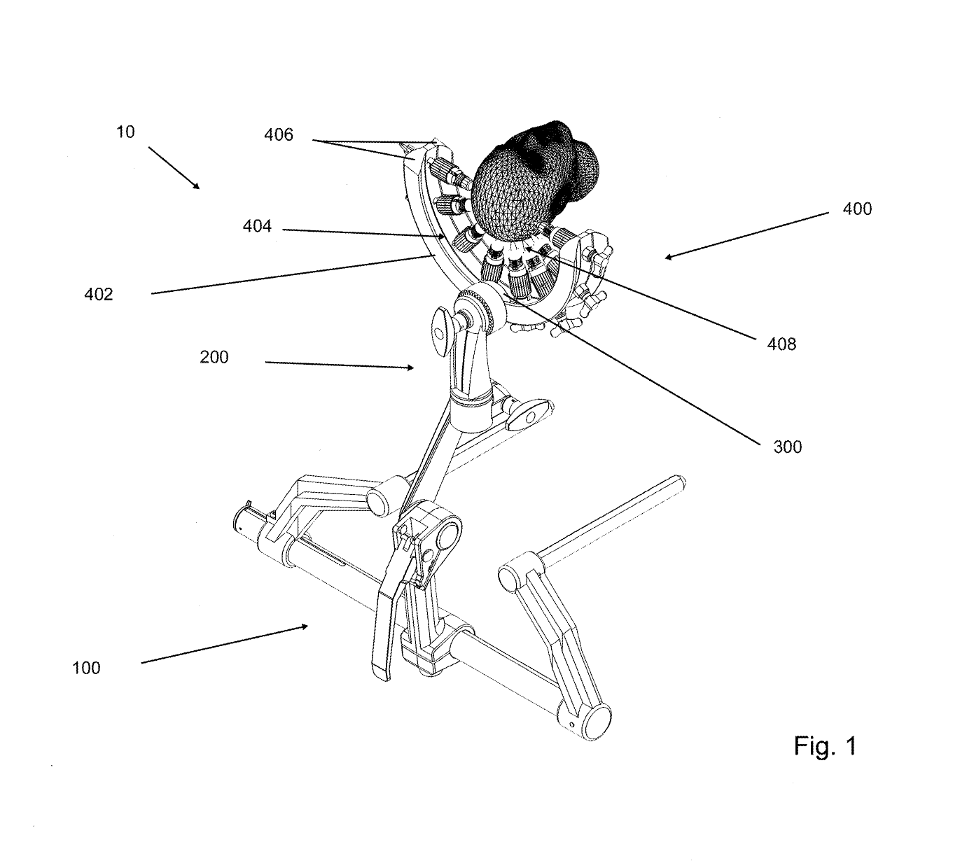

FIG. 1 depicts an exemplary patient stabilization system (10) connected with a mannequin head. The patient stabilization system (10) comprises a base unit (100), a swivel adapter (200), an interface adapter (300), and a HFD (400). The base unit (100) is configured to connect with a table or other patient support device. Connected with the base unit (100) is the swivel adapter (200). The swivel adapter (200) is configured to adjust the position of an attached HFD. In the present example, the HFD (400) attaches with the swivel adapter (200) by way of the interface adapter (300). Exemplary base units and swivel adapters may include, but not be limited to, those commercially available under the DORO brand name from pro med instruments GmbH.

The HFD (400) comprises a frame (402) and a plurality of stabilization assemblies (404). The frame (402) comprises a curved shape or arcuate shape and in the illustrated example a half-circle shape. In some versions, the frame (402) may extend such that it forms more than a half-circle, or less than a half-circle. The frame (402) further comprises a pair of rails (406) with one positioned along the front of the frame (402) and one positioned along the back of the frame (402). For the sake of directional reference, the front of the frame (402) shall be considered the part of the frame (402) closest to the swivel adapter (200), and the back of the frame (402) opposite the front. The rails (406) are configured to receive various accessories, e.g. retractor arms and others, that can connect with the rails (406), e.g. by a clamping action. While the present example describes a pair of rails (406), some other versions may be configured with a single rail on either the front side of the frame (402) or the back side of the frame (402).

As will be described in greater detail below, the stabilization assemblies (404) are configured to selectively connect with either pins (408) or pads (410) such that the HFD (400) setup can be modified as needed for use with all pins (408), all pads (410), or any combination of pins (408) and pads (410). In this fashion, the HFD (400) is interchangeable between an invasive setup using pins (408), a non-invasive setup using pads (410), or a combination invasive and non-invasive setup using some pins (408) and some pads (410).

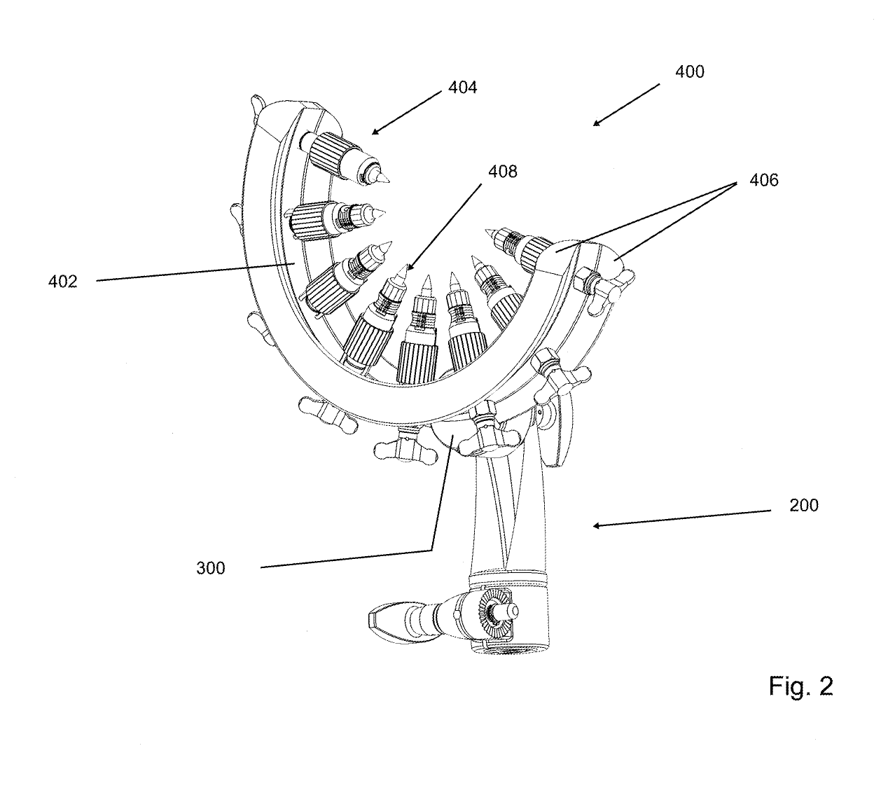

Referring to FIG. 2, the HFD (400) is shown with an invasive setup using all pins (408). In the present example, eight stabilization assemblies (404) are spaced along the inner curved surface of the frame (402) and connect with the frame (402). Of course in some other versions greater or fewer stabilization assemblies (404) may be used. Each of the stabilization assemblies (404) in the present example of FIG. 2 use a pin (408) connected with a distal end of the stabilization assembly (404). Each pin (408) is configured to contact a patient to stabilize at least a portion of the patient.

Furthermore, as illustrated in FIG. 2, the HFD (400) is shown adjusted rotationally relative to the interface adapter (300). As will be described in greater detail below, the interface adapter (300) connects with the swivel adapter (200) at one end, and connects with the HFD (400) at the other end. The connection with the HFD (400) is such that the interface adapter (300) attaches with one of the rails (406). This connection between the interface adapter (300) and the rail (406) is adjustable such that the frame (402) can selectively slide within the interface member (300). This adjustment allows the frame (402) to be adjusted rotationally so, for example, a patient's head positioned within the HFD (400) may be rotated to a desired position.

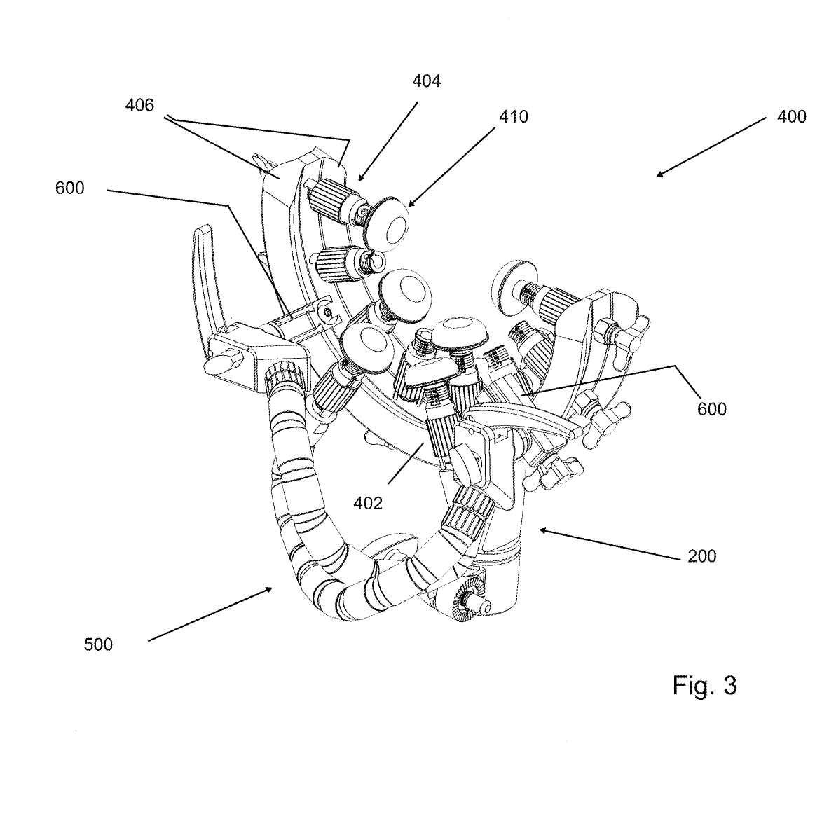

Referring now to FIG. 3, an alternate setup or configuration for the HFD (400) is shown where six pads (410) are used with the stabilization assemblies (404) instead of pins (408). Additionally, in the illustrated configuration of FIG. 3, two support arms (500) are attached with the rails (406) of the HFD (400), with each support arm (500) connected with a stabilization assembly (404), which further connects with a pad (410). The pads (410) can be gel pads in some versions, foam pads in some versions, rubber pads in some versions, or any other type of pad suitable to contact and support a patients head and/or face. Furthermore, while the present example illustrates round or circular shaped pads, other shapes may be used and can include, for example and not by way of limitation, horseshoe, straight, triangle, curved, among other shapes. With this configuration, the HFD (400) has a non-invasive setup or configuration. Such a configuration may be particularly suitable for pediatric patients, or patients with otherwise weak cranial bone structure.

In the present example, the support arms (500) connect with the rails (406) by way of clamp assemblies (600). The rails (406) comprise a dovetail shape and the clamp assemblies (600) have an inverse dovetail shape such that they securely mate with the rails (406). Exemplary clamp assemblies are described in U.S. Pat. Pub. 2013/0081636, published Apr. 4, 2013, entitled “Head Fixation Device and Apparatus for Securing Components Thereto,” the disclosure of which is incorporated by reference herein. In some other versions, other complementary profiles may be used for the rails (406) and clamp assemblies (600) other than the dovetail and inverse dovetail shapes. For instance, the rails (406) may have an I-beam shape with the clamp assemblies having an inverse I-beam shape. In other versions, the rails (406) may have a “T” shape with the clamp assemblies having an inverse “T” beam shape. When describing a shape as an inverse shape, it will be understood that the inverse shape means that the item possessing the inverse shape will have a void space with a cross sectional profile that matches the cross sectional profile of the rails (406) so that when joined there is a mating connection. In view of the teachings herein, other shapes for the rails (406) and clamp assemblies (600) will be apparent to those of ordinary skill in the art.

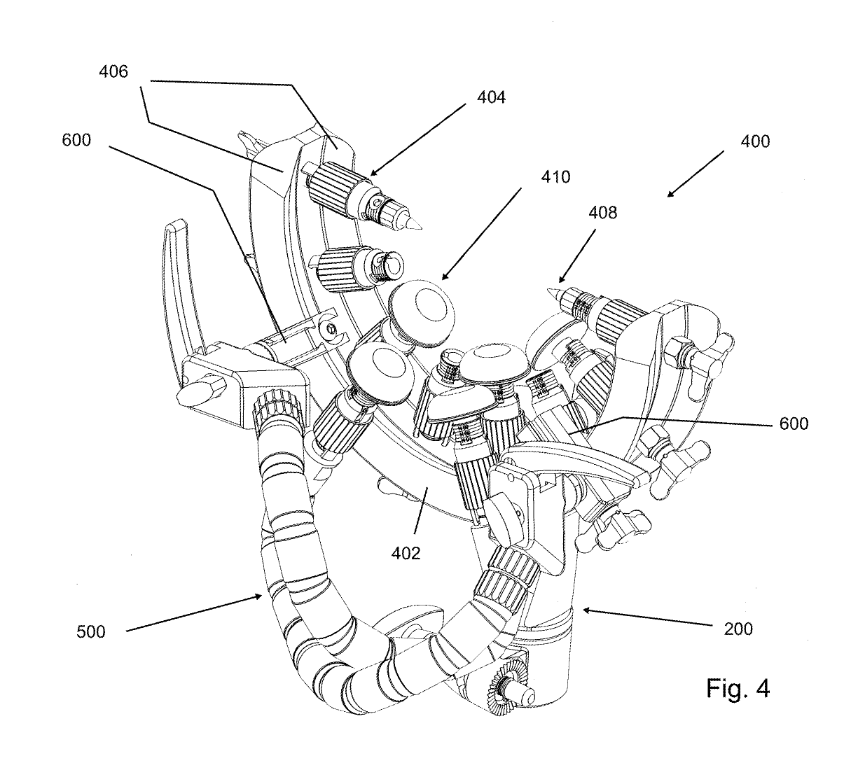

Referring now to FIG. 4, an alternate setup or configuration for the HFD (400) is shown where the two upper most pads (410) along the frame (402) are replaced with pins (408). In this configuration the HFD (400) is minimally invasive as the number of pins (408) used is minimized to at least some degree with pads (410) used instead in several locations. While the present example of FIG. 4 shows the pins (408) in the two upper most stabilization assemblies (404), this is not required in all versions. For instance, in other versions, the two pin locations shown in FIG. 4 can be changed to the location of other stabilization assemblies (404) as will be apparent to those of ordinary skill in the art in view of the teachings herein.

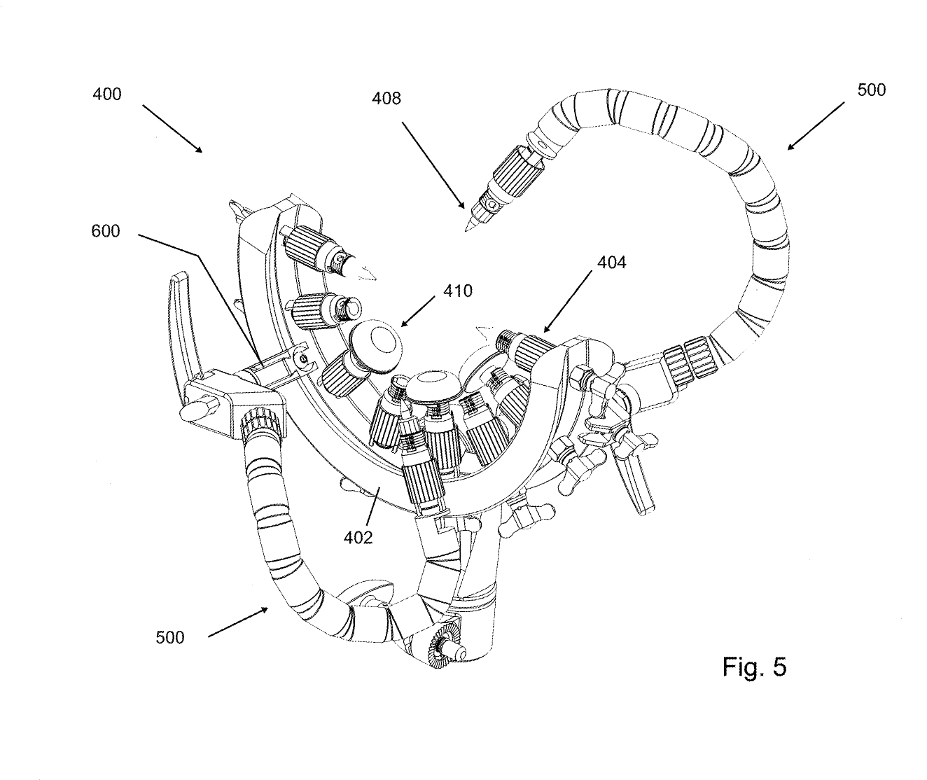

Referring now to FIG. 5, an alternate setup or configuration for the HFD (400) is shown similar to that shown in FIG. 4, but with the pads (410) on the support arms (500) replaced by pins (408). This setup or configuration provides another minimally invasive option, albeit more invasive than the configuration of FIG. 4.

While FIGS. 3-5 illustrate examples where the support arms (500) include the same type of stabilization feature—e.g. both pins (408) or both pads (410)—in some other versions one support arm (500) may use a pin (408) while another one of the support arms (500) may use a pad (410). Furthermore, while FIGS. 3-5 illustrate examples where two support arms (500) are used with the HFD (400), in other versions the support arms (500) are not required whatsoever. Still yet, in other versions a single support arm (500) or more than two support arms (500) may be used with the HFD (400). In view of the teachings herein the various configurations for the number and placement of pins (408) and/or pads (410) within the frame (402), as well as the number, placement, and stabilizing feature type used for the support arms (500) will be apparent to one of ordinary skill in the art.

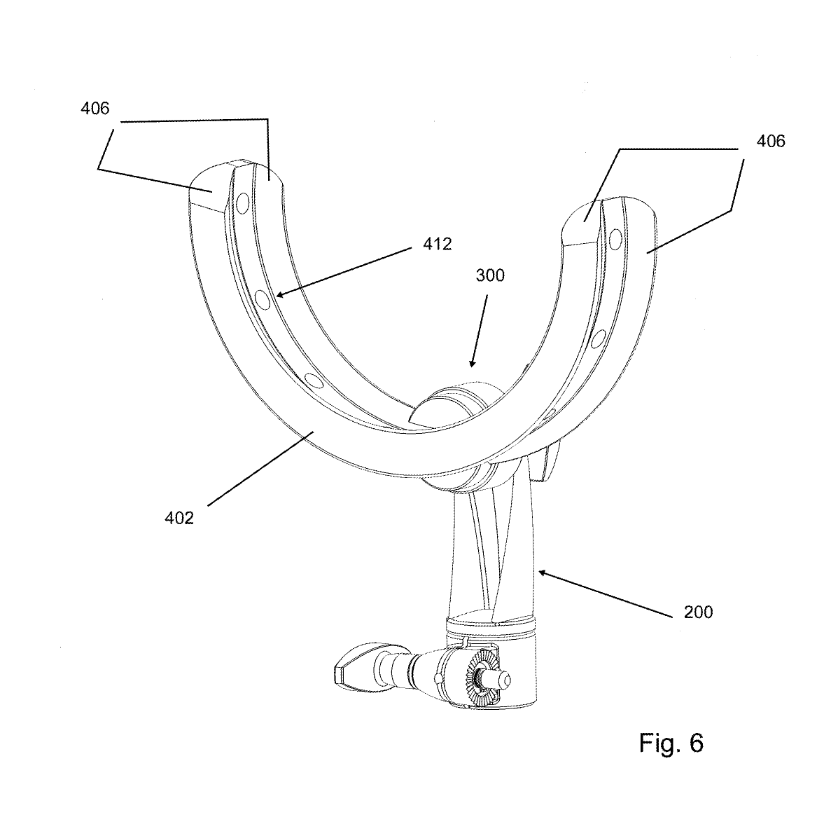

Referring now to FIG. 6, the frame (402) of the HFD (400) is shown without the stabilization assemblies (404) revealing a plurality of spaced bores (412) extending along the frame (402). As shown, the bores (412) are generally positioned in a central region of the frame (402) with the rails (406) on each side of the bores (412). As evident from the versions shown in FIGS. 1-5, the bores (412) are configured to receive the stabilization assemblies (404) in a selective manner. In view of the teachings herein, various ways to selectively connect the stabilization assemblies (404) with the bores (412) of the frame (402) will be apparent to those of ordinary skill in the art.

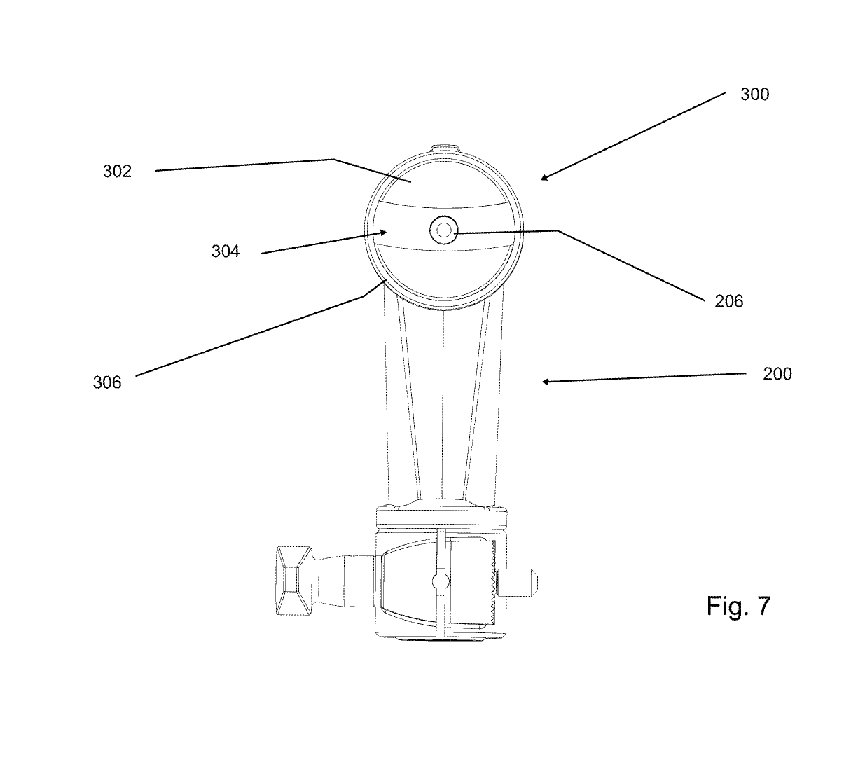

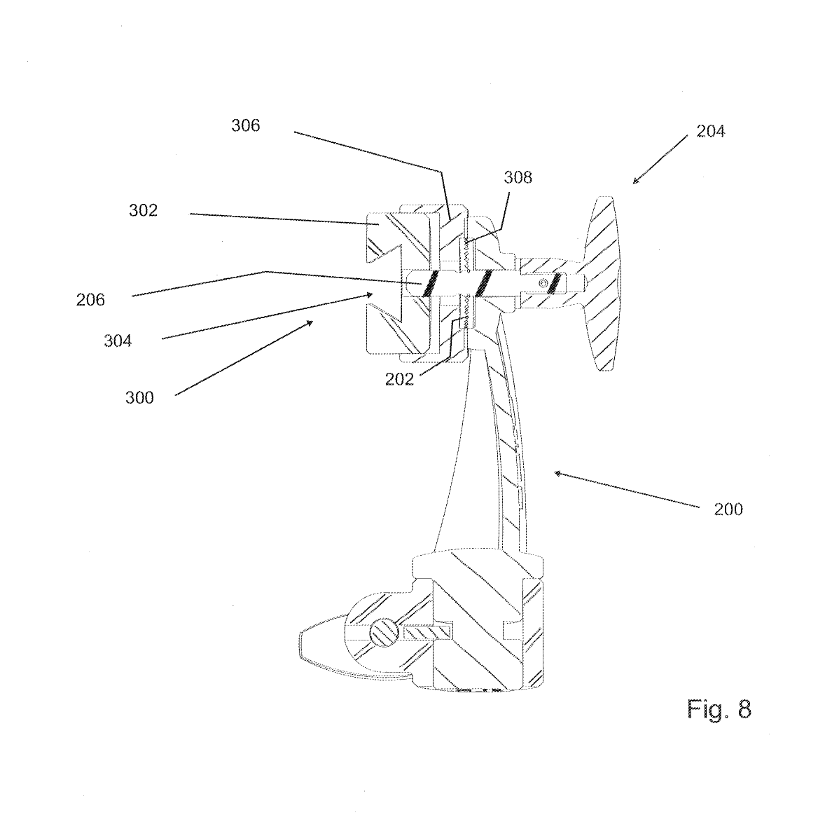

Referring now to FIGS. 7 and 8, the swivel adapter (200) is shown with the interface adapter (300) connected thereto. As seen in the front view of FIG. 7, the interface adapter (300) comprises a first member (302) that contacts and attaches with the rail (406) of the HFD (400). The first member (302) comprises a recess or slot (304) having an inverse dovetail shape—in other words where the recess or slot (304) represents a void space that in cross section has a dovetail shape of the same or similar proportions as the dovetail shape of the rail (406). With this configuration, the first member (302) can mate with the dovetail shape of the rail (406). Recess (304) may also be referred to as a groove or slot in some instances. Furthermore, the recess (304) is curved such that the curvature generally matches the curvature of the frame (402) and the rails (406) integrated with the frame (402). This curved configuration for the recess (304) enables the frame (402) of the HFD (400) to be rotated such that the frame (402) slides through the recess (304). In this manner the frame (402) and HFD (400) can be adjusted relative to the interface adapter (300), swivel adapter (200) and base unit (100). Other shapes for the recess (304) may be used where the rails (406) have profiles other than the dovetail shaped profile mentioned here, and such other shapes will be apparent to those of ordinary skill in the art in view of the teachings herein.

The interface adapter (300) further comprises a second member (306) that is configured to receive the first member (302). Both the first and second members (302, 306) have bores that align with one another. The second member (306) comprises a starburst interface (308). The swivel adapter (200) further comprises a starburst interface (202) and actuator (204) with screw (206). The screw (206) is configured to pass through the bore of the second member (306) and threadably engage with the threaded bore of the first member (302). In this fashion, the starburst interfaces (202, 308) can be drawn closer together to ultimately engage and secure the interface adapter (300) relative to the swivel adapter (200). In the reverse fashion, loosening the screw (206) creates separation between the starburst interfaces (202, 308) such that the relative position of the interface adapter (300) to swivel adapter (200) can be adjusted.

In the present example, the screw (206) also acts as a set-screw and can contact the frame (402) to secure the relative position of the frame (402) to the interface adapter (300). In the case where the frame (402) may be adjusted relative to the interface adapter (300), the actuator (204) may be rotated to loosen the screw (206) enough to rotatably slide the frame (402) through the recess (304) of the interface adapter (300). Once the frame (402) is in the desired position relative to the interface adapter (300) the actuator (204) can be tightened to securely hold the components in place.

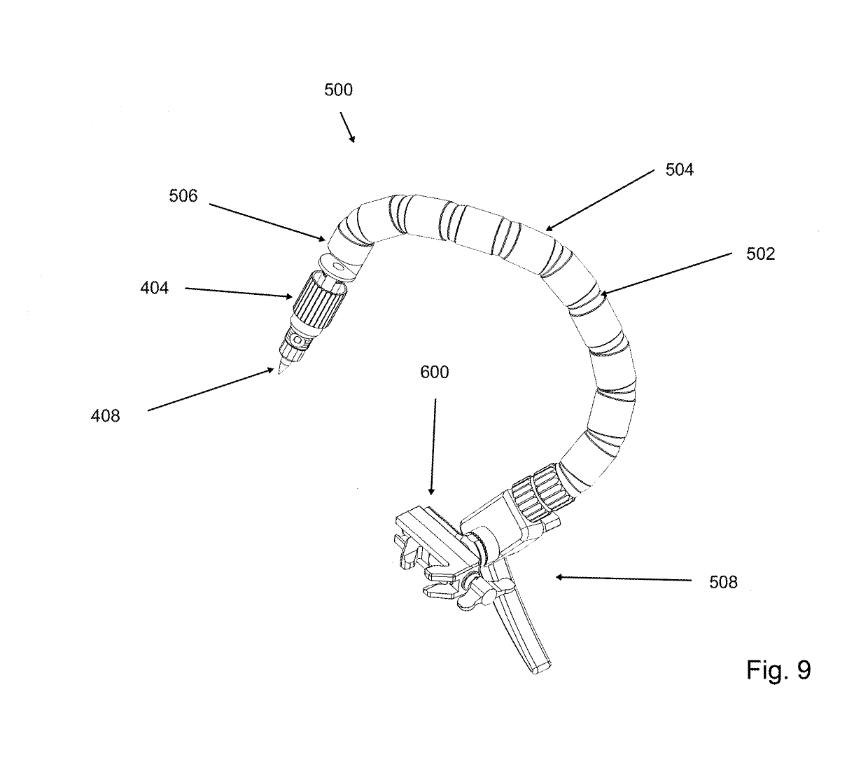

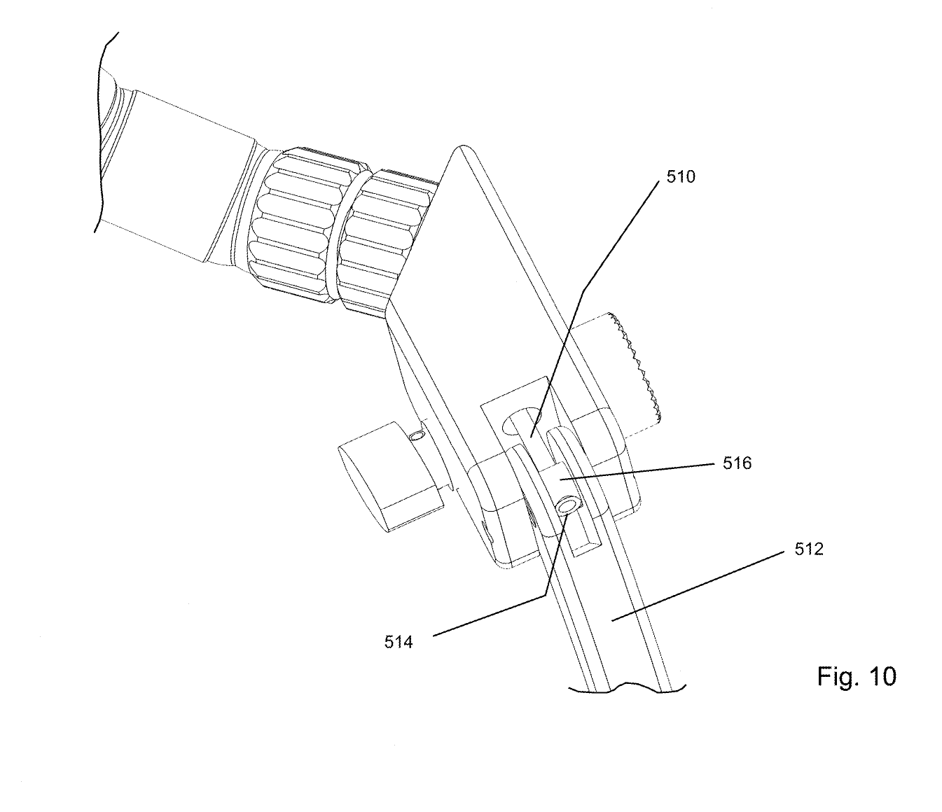

Referring now to FIGS. 9 and 10, the support arm (500) or portions thereof are shown. The support arms (500) comprise alternating double ball features (502) and sleeves (504). In some instances the double ball features (502) may be referred to as curved members while the sleeves (504) may be referred to as bushings. As shown in the present example, the alternating double ball features (502) and sleeves (504) make up the majority of the length of the support arm (500). It should be noted that the length of the support arms (500) may be greater or less than that shown in the illustrated versions.

At one end of the support arm (500) is an adapter (506) that connects with a stabilization assembly (404). As shown and described above, the stabilization assembly may hold either a pin (408) or pad (410) depending on the desired application. At the other end of the support arm (500) is an actuator (508) for controlling adjustment and positioning of the support arm (500). In the present example the support arm (500) comprises a cable (510) that extends from the actuator (508) to the other end of the support arm (500) where it connects with the adapter (506) or another feature of the support arm (500) near the adapter (506) end. At the actuator (508) end, the cable (510) connects with a lever (512), in the present example, by way of a threaded coupler (514) and holder (516).

Actuating the actuator (508) pulls or releases the cable (510) depending on the manner in which the actuator (508) is actuated. When the cable (510) is pulled or tightened, the double ball features (502) and sleeves (504) are drawn closer together and make contact such that the support arm (500) is locked in position. When the cable (510) is released or loosened, the reverse occurs and the support arm (500) is free to be manipulated and adjusted. While in the present example the actuator (508) comprises a configuration using a lever (512) to manipulate the cable (510), in other version the actuator (508) may comprise a different configuration such as a rotatable knob or other feature capable of manipulating the cable (510) to adjust the support arm (500).

In some instances, the support arm (500) has a configuration the same or similar to a flexible arm as described in U.S. Pat. Pub. 2014/0275799, published Sep. 18, 2014, entitled “Flexible Arm and Method of Using,” the disclosure of which is incorporated by reference herein. In some instances, the support arms (500) may be compared to retractor arms. Not required in all cases, but in some instances, compared with a retractor arm, the components of the support arms (500) may be wider and stiffer in order to bear more load. To this end, the cable (510) within the support arms (500) may be larger in diameter or stiffer compared to cables within standard retractor arms. Also, where standard retractor arms are generally used in the sterile field—over the surgical drapes—the support arms (500) are used in the non-sterile field—underneath surgical drapes. Of course, in other versions, the support arms (500) could be used in the sterile field.

Referring to FIG. 9, the actuator (508) of the support arm (500) includes an interface for connecting with the clamp assembly (600) so as to ultimately connect the support arm (500) with the frame (402) of the HFD (400) as discussed above. In the present example, the interface used for connecting the actuator (508) with the clamp assembly (600) is a pair of starburst interfaces. Moreover, using the clamp assembly (600) allows the support arm (500) to be adjustable along the frame (402) by sliding the support arm (500) along the rail (406) of the frame (402). Furthermore, this adjustment can occur with or without removing the support arm (500) from the frame (402), and with or without adjusting the tension within the support arm (500). In view of the teachings herein, other ways to connect the support arm (500) to the HFD (400) will be apparent to those of ordinary skill in the art.

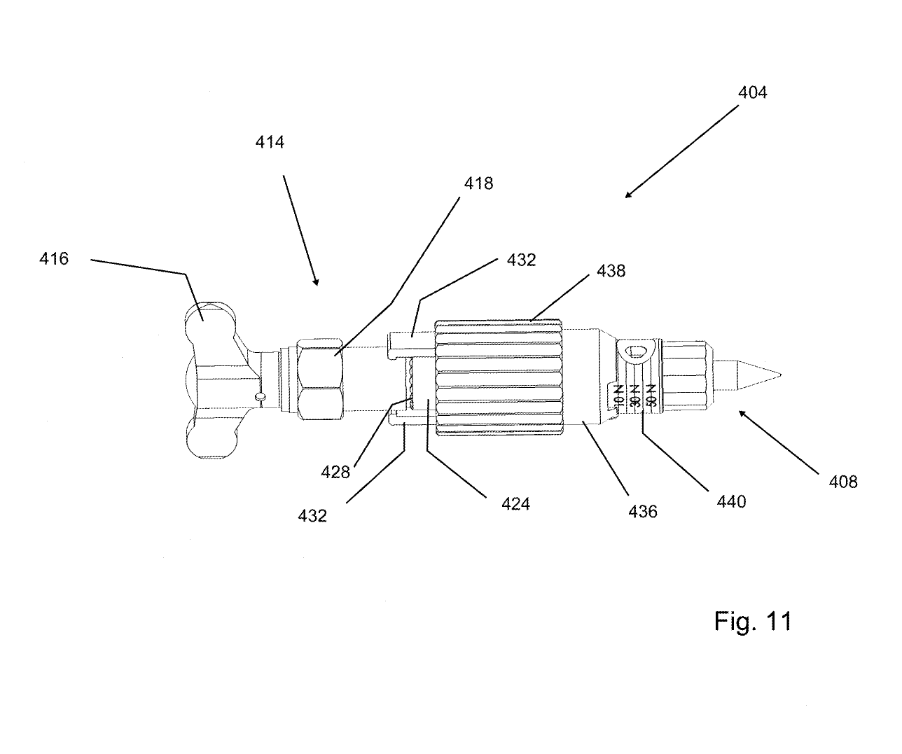

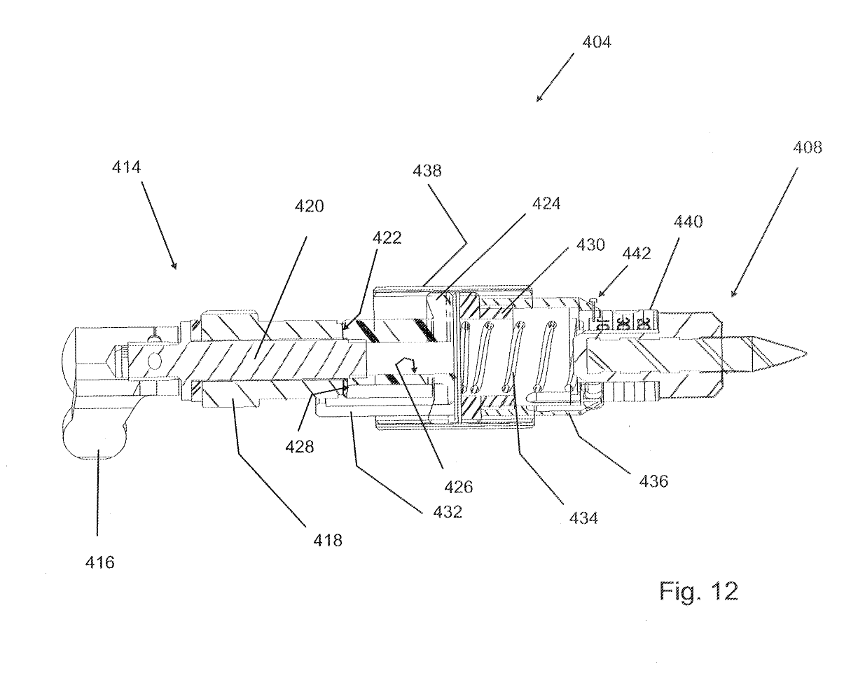

Referring now to FIGS. 11 and 12, the stabilization assemblies (404) are shown in greater detail. The stabilization assemblies (404) comprise an actuator (414) at one end that includes a knob (416), a nut (418), and a screw (420) or threaded rod.

The nut (418) has a bore extending through it such that the screw (420) extends through the nut (418). At the end of the nut (418) furthest from the knob (416) is a starburst interface (422).

Connecting with the screw (420) of the actuator (414) is a nut (424) having a threaded bore (426) such that the screw (420) threadably engages the threaded bore (426). Furthermore, when installing the stabilization assemblies (404) to the frame (402) of the HFD (400), the actuator (414) is positioned along the outer surface of the frame (402) with a portion of the nut (418) and screw (420) extending through the bore (412) in the frame (402). The remainder of the stabilization assembly (404) is positioned along the inner surface of the frame (402) with the nut (424) receiving the portion of the screw (420) extending through the bore (412) of the frame (402). The nut (424) comprises a starburst interface (428) that engages with the starburst interface (422) of the nut (418).

A collar (430) abuts one end of the nut (424) and includes a pair of feet (432) that extend proximally or toward the frame (402) when installed. When the stabilization assembly (404) is installed on the frame (402), the feet (432) contact the inner surface of the frame (402). A spring (434) is positioned against one end of the collar (430) in a center region of the collar (430). Surrounding the spring (434) and positioned against an outer region of the collar (430) is a sleeve (436). Surrounding portions of the nut (424), the collar (430), the spring (434), and the sleeve (436) is an outer sleeve (438).

The sleeve (436) has an opening extending through it and configured to receive an indicating member (440) that contacts the spring (434). Within the indicating member (440), either a pin (408) or pad (410) is received depending on the application. The pin (408) or pad (410) abut the indicating member (440) or connect with the indicating member (440), e.g. by way of a screw, etc., such that force exerted on the pin (408) or pad (410)—e.g. from contacting a patient's head—causes the indicating member (440) to push on the spring (434). The indicating member (440) includes markings that may be printed or engraved on the indicating member (440) and such markings indicate force increments or levels. In use, when the pin (408) or pad (410) is positioned and holding a patient's head, the force exerted on the patient's head is indicated on the indicating member (440) where the end of the sleeve (436) closest to the pin (408) or pad (410) aligns with the force markings on the indicating member (440). The spring force of spring (434) is known such that accurate force indications can be obtained.

Adjustment of the force on the patient can be achieved by actuating the actuator (414)—e.g. by turning the knob (416) to either increase the force or decrease the force. For instance, when the knob (416) is rotated to increase the force, the threaded engagement of the screw (420) with the nut (424) causes the nut (424) to translate toward the patient and in the process the nut (424) drives the collar (430), which in turn compresses the spring (434). When the knob (416) is rotated to decrease the force, the reverse operations occur.

In some versions, one or more of the stabilizing assemblies (404) can include an optional locking screw configured to fix or hold the pressure setting at a desired level. In one such example, once the patient is positioned and stabilized at the desired pressure, the locking screw can be inserted and/or tightened such that the pressure is fixed. For example, in one version using a locking screw (442), the locking screw (442) securely connects the indicating member (440) to the sleeve (436) such that the indicating member (440) is stationary or locked in position relative to the sleeve (436). With the indicating member (440) locked, and with the feet (432) contacting the frame (402), the force imparted to the patient remains fixed. In view of the teachings herein, other ways to hold the pressure at a constant level will be apparent to those of ordinary skill in the art.

As described above, it should be noted that the stabilizing assemblies (404) are adaptable to retain either pins (408) or pads (410). Furthermore, the stabilizing assemblies (404) include a pressure scale feature to provide an indication of the force being applied to the patient. Additionally, the stabilizing assemblies (404) are usable or connectable with the frame (402) directly, or with a support arm (500). Also, the rails (406) of the HFD (400) are configured to receive other accessories, e.g. brain retractors etc., in addition to or in lieu of the support arms (500).

It should be understood that any one or more of the teachings, expressions, embodiments, examples, etc. disclosed herein may be combined with any one or more of the other teachings, expressions, embodiments, examples, etc. that are disclosed herein. The teachings, expressions, embodiments, examples, etc. disclosed herein should therefore not be viewed in isolation relative to each other. Various suitable ways in which numerous aspects of the present disclosure may be combined will be readily apparent to those of ordinary skill in the art in view of the teachings disclosed herein. Such modifications and variations are intended to be included within the scope of both the present disclosure and the claims.

Having shown and described various embodiments of the present disclosure, further adaptations of the methods and systems described herein may be accomplished by appropriate modifications by one of ordinary skill in the art without departing from the scope of the present disclosure. Several of such potential modifications have been mentioned, and others will be apparent to those skilled in the art. For instance, examples, embodiments, geometrics, materials, dimensions, ratios, steps, and the like discussed above are illustrative and are not required. Accordingly, the scope of the present disclosure should be considered in terms of the following claims and is understood not to be limited to the details of structure and operation shown and described in the specification and drawings.

I/We claim:

1. A head fixation device for use to stabilize a patient, the head fixation device comprising:
a. a curved frame having a first rail;
b. a plurality of stabilizing assemblies connectable to the frame; and
c. a plurality of stabilizing features each connectable to one of the plurality of stabilizing assemblies, wherein changing a type of one or more of the plurality of stabilizing features enables the head fixation device to change configuration from invasive to non-invasive or minimally invasive.

2. The device of claim 1, wherein the curved frame comprises about a half circle configuration.

3. The device of claim 1, wherein the frame comprises a second rail.

4. The device of claim 3, wherein the first rail is located on a front side of the frame and wherein the second rail is located on a back side of the frame.

5. The device of claim 1, wherein the frame comprises a plurality of bores, wherein each of the plurality of bores are configured to receive one of the plurality of stabilizing assemblies.

6. The device of claim 1, further comprising a support arm connectable to the frame, wherein the support arm includes one of the plurality of stabilizing assemblies.

7. The device of claim 6, further comprising more than one support arm.

8. The device of claim 1, wherein each one of the plurality of stabilizing features comprises a select one of a pin and a pad.

9. The device of claim 1, wherein the frame is adjustably connectable to an adapter, wherein the adapter is connectable to a patient support device.

10. The device of claim 9, wherein the device is rotatable relative to the adapter.

11. The device of claim 10, wherein the adapter comprises a recess configured to receive the first rail of the frame.

12. The device of claim 11, wherein the recess is curved to match a curvature of the frame.

13. The device of claim 11, wherein the first rail is selectively slidable along the recess of the adapter.

14. The device of claim 1, wherein the plurality of stabilizing assemblies is configured to adjust a magnitude of force applied during use.

15. The device of claim 14, wherein the plurality of stabilizing assemblies comprises an indicator member configured to indicate the force applied during use.

16. The device of claim 15, wherein the plurality of stabilizing assemblies further comprises an actuator and a spring, wherein the force applied during use is adjustable using the actuator to adjust compression of the spring.

17. The device of claim 16, wherein a select one or more of the plurality of stabilizing assemblies further comprises a locking screw configured to set the force applied during use.

18. A head fixation device for use to stabilize a patient, the head fixation device comprising:
a. a frame;
b. a first stabilizing assembly directly connectable to the frame, wherein the first stabilizing assembly comprises an indicator member configured to indicate a force applied during use, wherein the first stabilizing assemblies is configured to adjust the of force applied during use;
c. a first stabilizing feature connectable to the first stabilizing assembly, wherein the first stabilizing feature comprises a select one of a first pin and a first pad; and
d. a support arm connectable to the frame, wherein the support arm includes a second stabilizing assembly and a second stabilizing feature connectable to the second stabilizing assembly, wherein the second stabilizing feature comprises a select one of a second pin and a second pad.

19. The device of claim 18, wherein the first stabilizing feature and the second stabilizing feature provide a minimally invasive configuration for the device.

20. The device of claim 18, wherein the frame comprises a pair of rails, wherein the support arm is connectable to the frame about a select one of the pair of rails.
